(19) United States
(12) Patent Application Publication
(10) Pub. No.: US 2020/0297972 A1
(43) Pub. Date: Sep. 24, 2020

(54) CATHETER WITH SEAMLESS FLEXIBILITY TRANSITIONS

(71) Applicant: Imperative Care, Inc., Campbell, CA (US)

(72) Inventors: Brandon Yee, Oakland, CA (US); Ashoor Shahbazi Yourgenlow, San Jose, CA (US); Yi Yang, San Francisco, CA (US); Michael Luna, San Jose, CA (US)

(21) Appl. No.: 16/802,317

(22) Filed: Feb. 26, 2020

Related U.S. Application Data
(60) Provisional application No. 62/811,308, filed on Feb. 27, 2019.

(51) Int. Cl. A61M 25/01; A61M 25/00

(52) U.S. Cl. CPC A61M 25/0138; A61M 25/0053; A61M 2025/0059; A61M 25/008; A61M 25/0054

(57) ABSTRACT

Disclosed is a tubular wire coil in which the wire varies in diameter over the length of the coil. The coil can be used to create a coiled reinforcement member for a tubular body, having different physical properties along the length of the body without the need for welds or joints to connect different wire segments. The coil can thus reduce unwanted catheter tip or welded joint stiffness without sacrificing desired proximal stiffness in, for example, a catheter such as a neurovascular catheter.

### CROSS-REFERENCE TO RELATED APPLICATIONS

This application claims the priority benefit under 35 U.S.C. § 119(e) of U.S. Provisional Application No. 62/811,308, filed Feb. 27, 2019, the entirety of which is hereby incorporated by reference herein.

### BACKGROUND OF THE INVENTION

In a various applications, a tubular body is provided with a reinforcing feature to achieve or optimize a balance among any of a variety of performance characteristics. In the context of medical transvascular catheters, these characteristics include pushability (column strength), torque ability, lateral flexibility and kink resistance among others.

Catheters configured to reach remote, tortuous, vasculature such as in the brain are preferably configured with differing characteristics along their axial length. This is sometimes accomplished by providing reinforcement components within the catheter side wall such as spring coils having different flexibility and attaching them end to end with a successive increase in flexibility in a distal direction. Unfortunately, the joint between adjacent segments can leave an irregularity such as a stiff spot or stiff distal tip, which may adversely affect catheter performance.

Thus, there remains a need for a tubular support for use in catheter or other tubular body fabrication, that allows varying flexibility along the axial length of the tubular body but exhibits an improved flexibility profile.

### SUMMARY OF THE INVENTION

Disclosed is a coil formed from a single wire that varies in diameter over the length of the coil. The wire can be used to create a coiled reinforcement member for a catheter. The reinforcement member can reduce unwanted catheter tip or welded joint stiffness without sacrificing desired proximal stiffness. In one implementation the wire comprises Nitinol.

In accordance with one aspect of the present invention, there is provided a catheter having jointless transitions in flexibility. The catheter comprises an elongate, flexible tubular body, having a proximal end, a distal end and a tubular side wall defining at least one lumen extending axially therethrough. The side wall includes a helical wire coil, the coil having a first, proximal zone in which the wire has a first diameter separated by a transition from a second, relatively distal zone in which the wire has a second, smaller diameter. The first zone, the transition and the second zone are formed from a single, continuous wire without the need for welds or other joints.

The transition may have an axial length of no more than about 1 cm or no more than about 0.5 cm or 2 mm or less. The lumen may have the same inside diameter in the proximal zone and the distal zone. The transition may be located within about 5 cm or within about 2 cm or 1 cm or less from the distal end of the catheter.

The catheter may have two or three or more zones, with at least a jointless transition between each adjacent pair of zones. The wire in the first, proximal zone may have a diameter that is at least about 0.0002 inches greater than the diameter of the wire in the second zone, and in some implementations the wire in the first zone has a diameter that is at least about 0.0004 inches greater than the diameter of the wire in the second zone.

The catheter may be formed by a process including winding a wire to form a tubular body coil, and thereafter exposing a portion of the tubular body to a pickling agent. The process may alternatively comprise the steps of exposing a portion of a wire to a pickling agent, and thereafter winding the wire into a tubular coil body. The pickling agent may comprise an acid solution.

### BRIEF DESCRIPTION OF THE DRAWINGS

FIG. 1 is a side elevational cross section through a catheter having seamless flexibility transitions.

FIGS. 1A and 1B are enlargements of flexibility transitions in the catheter of FIG. 1.

FIG.1C is a side elevational cross section through a three zone, two transition catheter shaft.

FIG. 2 illustrates a cross-sectional elevational view of a catheter wall according to another embodiment.

FIG. 3A illustrates a cross-sectional elevational view of a catheter wall according to another embodiment, showing one or more axially extending filaments.

FIG. 3B describes a side elevational view of the catheter of FIG. 3A

FIG. 3C illustrates a cross-sectional view taken along the line C-C of FIG. 3B, showing one or more axially extending filaments.

FIG. 3D is a side elevational cross section through an angled distal catheter or extension tube tip.

FIG. 4A depicts a side elevational view of a catheter according to one embodiment.

FIG. 4B describes a cross-sectional elevational view taken along the line A-A of FIG. 4A.

FIG. 4C illustrates a cross-sectional view taken along the line B-B of FIG. 4A.

FIG. 5A depicts a side elevational view of a catheter according to another embodiment.

FIG. 5B describes a cross-sectional elevational view taken along the line A-A of FIG. 5A, showing one or more axially extending filaments.

FIG. 5C illustrates a cross-sectional view taken along the line B-B of FIG. 5A, showing one or more axially extending filaments.

FIG. 6A illustrates a side elevational view of a progressively enhanced flexibility catheter according to an embodiment.

FIG. 6B is a proximal end view of the enhanced flexibility catheter of FIG. 6A.

### DETAILED DESCRIPTION OF THE PREFERRED EMBODIMENT

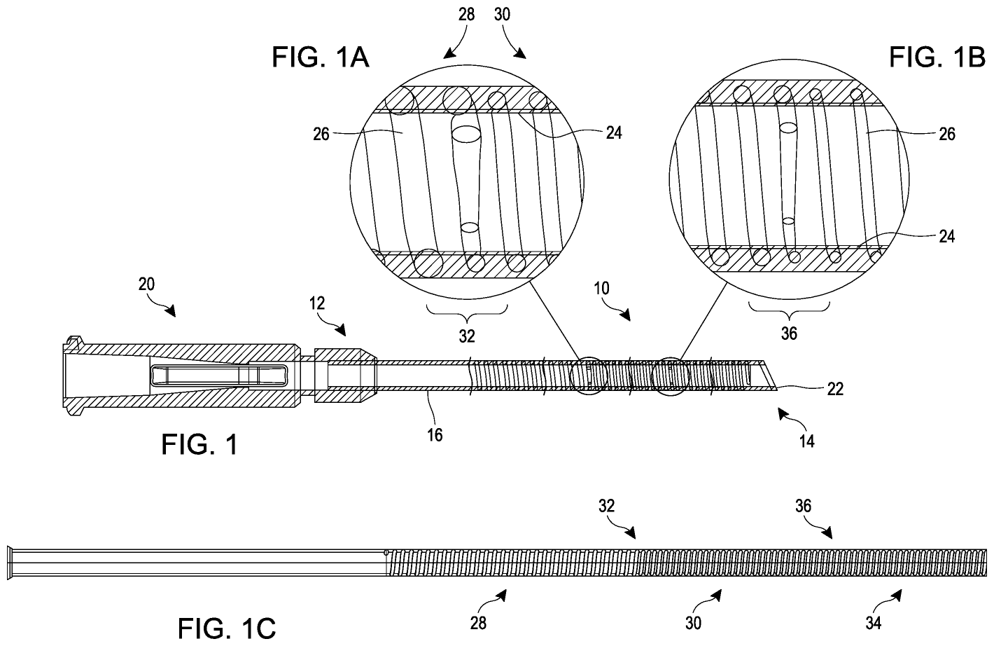

Referring to FIG. 1, there is disclosed a catheter 10 in accordance with one aspect of the present invention. Although primarily described in the context of an aspiration catheter with a single central lumen, catheters of the present invention can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the present invention will be described primarily in the context of removing obstructive material from remote vasculature in the brain, but has applicability as an access catheter for delivery and removal of any of a variety of diagnostics or therapeutic devices with or without aspiration. In addition, coils and wires disclosed herein may be used in any of a variety of non medical applications, such as flexible tueing or other structures where a stepped flexibility may be desired.

The catheters disclosed herein may readily be adapted for use throughout the body wherever it may be desirable to distally advance a low profile high flexibility catheter into small and/or tortuous vasculature. For example, catheter shafts in accordance with the present invention may be dimensioned for use throughout the coronary and peripheral vasculature, the gastrointestinal tract, the urethra, ureters, Fallopian tubes and other lumens and potential lumens, as well. The catheter shaft construction of the present invention may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants.

The catheter 10 generally comprises an elongate tubular body 16 extending between a proximal end 12 and a distal functional end 14. The length of the tubular body 16 depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site, as discussed in further detail below.

Catheters in accordance with the present invention will have a length and diameter suitable for the intended access point and target location. In one example, referring to FIG. 1, the catheter 10 may have an effective length from the manifold 20 to distal tip 22 generally no more than about 180 cm or no more than about 160 cm. and typically from about 70 cm to about 150 cm, from about 90 cm to about 130 cm, or from about 105 cm to about 115 cm. The outer diameter of the catheter 10 may be from about 0.035 inches to about 0.15 inches, from about 0.09 inches to about 0.13 inches, and may be lower in a distal segment than in a proximal segment.

The inner diameter of the catheter 10 in a single central lumen embodiment may be greater than or equal to about 0.1 inches, greater than or equal to about 0.088 inches, or greater than or equal to about 0.08 inches, or greater than or equal to about 0.06. The inner diameter of the catheter 10 in a single central lumen embodiment may be less than about 0.20 inches or 0.15 inches, or less than or equal to about 0.11 inches, less than or equal to about 0.1 inches, less than or equal to about 0.088 inches, or less than or equal to about 0.07 inches, and often no more than about 0.095 inches.

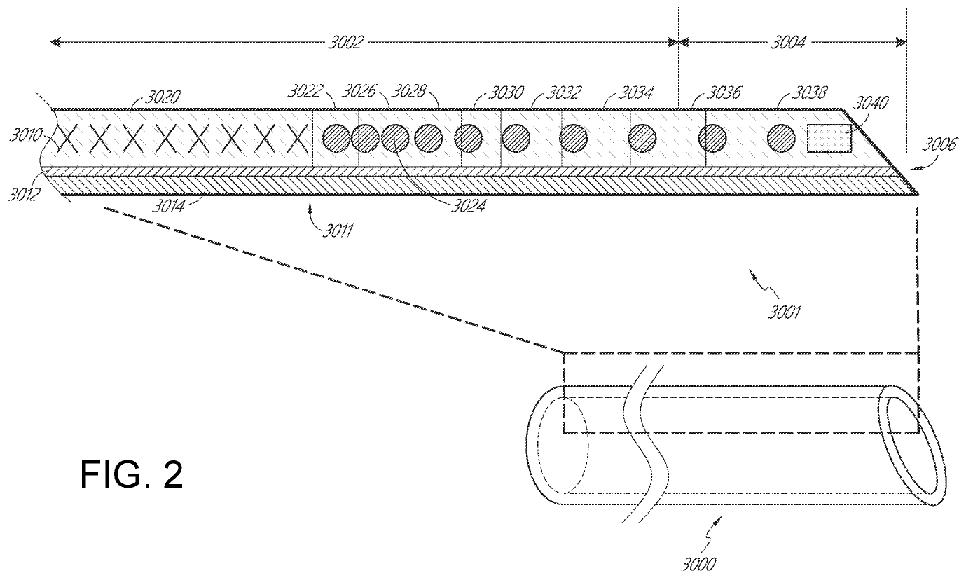

Referring to FIGS. 1A and 1B, an inner liner 24 may be formed by dip coating a mandrel (not shown) to provide a thin walled tubular inside layer of the catheter body 16. The dip coating may be produced by coating a wire such as a silver coated copper wire in PTFE. The mandrel may later in the process be axially elongated to reduce diameter, and removed to leave the tubular inner liner. The outside surface of the tubular inner liner 24 may thereafter be coated with a soft tie layer 3012 (see, e.g. FIG. 2) such as polyurethane (e.g., Tecoflex™), to produce a layer having a thickness of no more than about 0.005 inches, and in some implementations approximately 0.001 inches. The tie layer will generally extend along at least about the most distal 10 cm or 20 cm of the catheter shaft 10 generally less than about 50 cm and may in one implementation extend approximately the distal 30 cm of the catheter body 16.

A helical coil may thereafter be wound around the adjacent inner tubular layer to provide radial support and kink resistance while also regulating flexibility of the final construct. Preferably, the coil comprises at least a first, relatively proximal zone having a first flexibility. A distal end of the first zone joins a proximal end of a second zone having a second typically greater flexibility, at a first transition between the two zones. Preferably, the first and second zones are continuous across the transition, having been formed from a single continuous wire.

A third zone may be provided, distally of the second zone and separated from the second zone by a second transition. All three zones and two transitions may be formed from a single continuous wire. The diameter of the wire in the first zone is greater than the diameter of the wire in the second zone. In a three zone embodiment, the diameter of the wire in the third, distal most zone is lower than the diameter of the wire in the proximally adjacent, second zone. Thus, the diameter of the wire from which the coil is wound steps down or ramps down in a proximal to distal direction.

The wire in any given zone may have a substantially constant diameter throughout the zone, so flexibility is ‘stepped’ up at each transition along the length of the catheter. One or more zones can alternatively have a continuous, graduated change in flexibility from one end to the other, such as by regulating the rate of withdrawal from acid bath immersion as is discussed below. Typically, each successive zone will increase in flexibility in a distal direction.

Referring to FIG. 1C, the diameter transitions from a first, greater cross section in a proximal zone 28 to a second, smaller cross section in a relatively distal zone 30, separated by transition 32. A third zone 34 is separated from second zone 30 by a second transition 36. The wire in the proximal zone 28, middle zone 30, distal zone 34 and transitions 32 and 36 are continuous, without welds or other connecting structures.

As used herein, ‘wire’ includes both round and non round (e.g., rectangular) cross section, and ‘diameter’ includes cross section in the radial direction for non round wires.

In a construction where the coil is wound around the tie layer as described above, the transition(s) may be formed in the wire prior to winding the coil around the tubular tie layer such as by exposing the wire to a subtractive process. Alternatively, a constant diameter wire may be wound into a coil which is removed from the mandrel and a distal section of the coil exposed to a subtractive process to reduce the diameter of the wire. The coil can thereafter be fabricated into a catheter side wall in accordance with known techniques.

In one implementation of a neurovascular catheter, the wire in the first, proximal zone has a diameter of about 0.0030 inches, and transitions to a diameter of about 0.025 at the first transition 32. A relatively distal third zone (second or third or fourth or more zone in a multi zone embodiment) may have a wire diameter of about 0.020 inches. Stated diameters are the average diameter of the wire within the zone.

The axial length of the distal most zone is no more than about 5 cm, typically no more than about 2.5 cm or 2.0 cm or less to provide a highly flexible distal tip. In a three zone embodiment, the first transition will typically be spaced at least about 5 cm and potentially at least about 10 cm or 20 cm or more but less than about 80 cm or less than about 50 cm from the distal most transition, depending upon the particular target anatomy and desired column strength and flexibility profile of the catheter.

A reduction in the wire diameter across a transition will preferably decrease the catheter stiffness by at least about 10% or at least about 15% or 20% or more compared to the proximally adjacent zone.

One subtractive process for creating a stepped diameter wire may include chemical etching or pickling to uniformly remove material around the wire diameter around select desired locations/lengths (hence selectively reducing the diameter of a target zone) with an acid solution. For example, reduction of a Nitinol wire may be accomplished by exposure (e.g., immersion) in a solution such as, but not limited to, the following: a) Sulfamic Acid, Hydrogen Peroxide, and DI water; b) Hydrofluoric, Nitric acid and DI water; and/or c) Sulfuric acid. In one implementation, a portion of a length of a Nitinol wire coil was immersed in a solution comprising Sulfamic Acid, Hydrogen Peroxide, and DI water at 37 degree C. for approximately 72 hrs. The measured diameter of the coil was reduced from approximately 0.00254 inches to approximately 0.00198 inches by the procedure. In some embodiments, the processes described herein may be used on materials other than nitinol, such as stainless steel.

FIG. 2 illustrates a cross section through the sidewall of a distal portion of a single lumen catheter, that may be fabricated using the stepped coil disclosed herein. Adjacent loops or filars of the coil 3024 may have a constant pitch throughout the length of the coil (see FIG. 1C) or may be closely tightly wound in a proximal zone with a distal section having looser spacing between adjacent loops. In an embodiment having a coil section 3024 with an axial length of at least between about 20% and 30% of the overall catheter length, (e.g., 28 cm coil length in a 110 cm catheter shaft 16), at least the distal 1 or 2 or 3 or 4 cm of the coil will have a spacing that is at least about 130%, and in some implementations at least about 150% or more than the spacing in the proximal coil section. In a 110 cm catheter shaft 3000 having a Nitinol coil the spacing in the proximal coil may be about 0.004 inches and in the distal section may be at least about 0.006 inches or 0.007 inches or more.

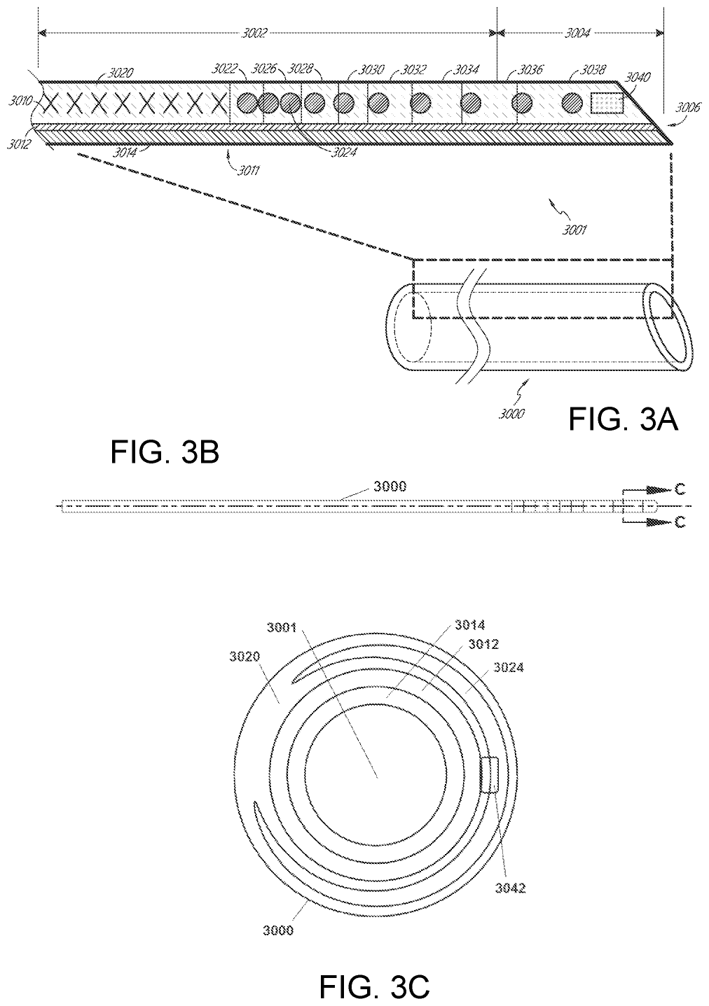

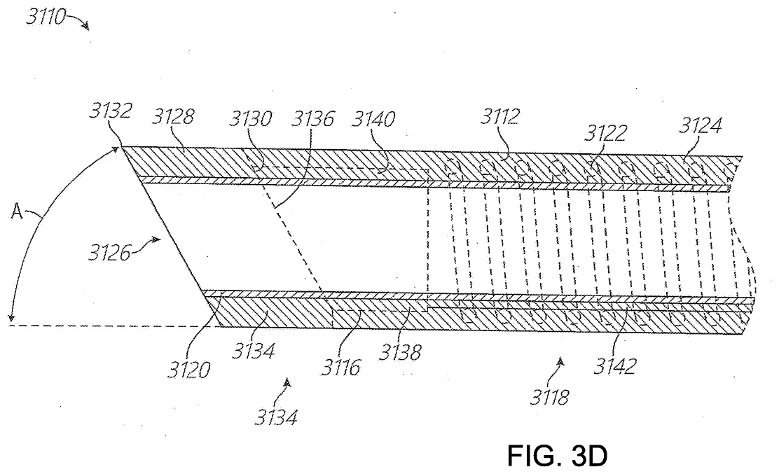

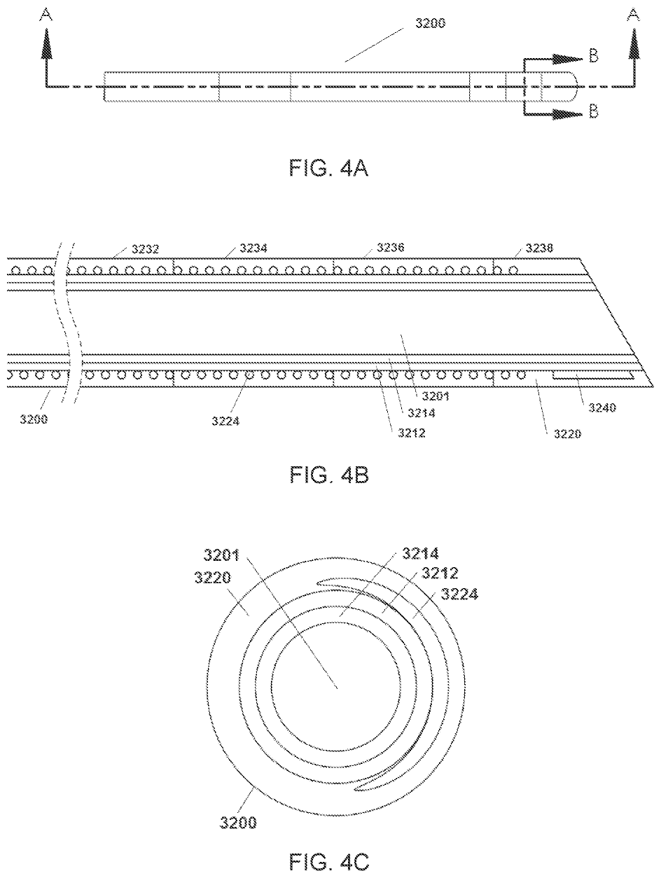

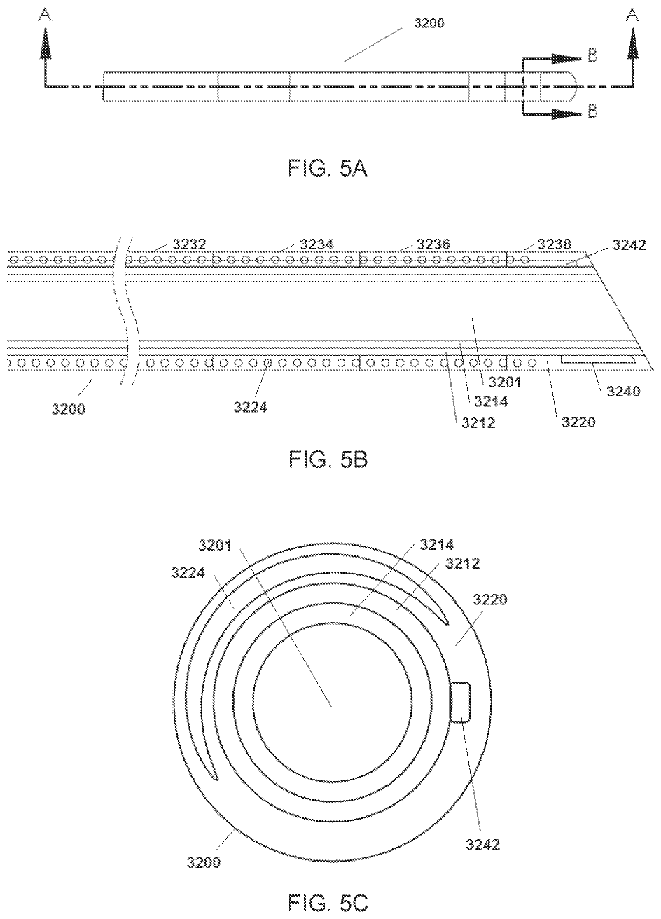

The distal end of the coil 3024 can be spaced proximally from the distal end of the inner liner 3014, for example, to provide room for an annular radiopaque marker 3040. The coil 3024 may be set back proximally from the distal end, in some embodiments, by approximately no more than 1 cm, 2 cm, or 3 cm. In one embodiment, the distal end of the catheter 10 is provided with a beveled distal surface 3006 residing on a plane having an angle of at least about 10° or 20° and in one embodiment about 30° with respect to a longitudinal axis of the catheter 10. The radiopaque marker 3040 may reside in a plane that is transverse to the longitudinal axis. Alternatively, at least the distally facing edge of the annular radiopaque marker 3040 may be an ellipse, residing on a plane which is inclined with respect to the longitudinal axis to complement the bevel angle of the distal surface 3006. Additional details are described in connection with FIG. 3D below.

After applying the proximal braid 3010, the distal coil 3024 and the RO marker 3040 an outer jacket 3020 maybe applied such as a shrink wrap tube to enclose the catheter body 16. The outer shrink-wrapped sleeve 3020 may comprise any of a variety of materials, such as polyethylene, polyurethane, polyether block amide (e.g., PEBAX™), nylon or others known in the art. Sufficient heat is applied to cause the polymer to flow into and embed the proximal braid and distal coil.

In one implementation, the outer shrink wrap jacket 3020 is formed by sequentially advancing a plurality of short tubular segments 3022, 3026, 3028, 3030, 3032, 3034, 3036, 3038 concentrically over the catheter shaft subassembly, and applying heat to shrink the sections on to the catheter 10 and provide a smooth continuous outer tubular body. The foregoing construction may extend along at least the most distal 10 cm, and preferably at least about the most distal 20 cm, 25 cm, 30 cm, 35 cm, 40 cm, or more than 40 cm of the catheter body 10. The entire length of the outer shrink wrap jacket 3020 may be formed from tubular segments and the length of the distal tubular segments (e.g., 3022, 3026, 3028, 3030, 3032, 3034, 3036, 3038) may be shorter than the one or more tubular segments forming the proximal portion of the outer shrink wrap jacket 3020 in order to provide steeper transitions in flexibility toward the distal end of the catheter 10.

The durometer of the outer wall segments may decrease in a distal direction. For example, proximal segments such as 3022 and 3026, may have a durometer of at least about 60 or 70D, with gradual decrease in durometer of successive segments in a distal direction to a durometer of no more than about 35D or 25D or lower. A 25 cm section may have at least about 3 or 5 or 7 or more segments and the catheter 10 overall may have at least about 6 or 8 or 10 or more distinct flexibility zones. The distal 1 or 2 or 4 or more segments 3036, 3038, may have a smaller OD following shrinking than the more proximal segments 3022-3034 to produce a step down in OD for the finished catheter body 16. The length of the lower OD section 3004 may be within the range of from about 3 cm to about 15 cm and in some embodiments is within the range of from about 5 cm to about 10 cm such as about 7 or 8 cm, and may be accomplished by providing the distal segments 3036, 3038 with a lower wall thickness.

Referring to FIGS. 3A-3C, the catheter may further comprise a tension support for increasing the tension resistance in the distal zone. The tension support may comprise a filament and, more specifically, may comprise one or more axially extending mono strand or multi strand filaments 3042. The one or more axially extending filaments 3042 may be axially placed inside the catheter wall near the distal end of the catheter. The one or more axially extending filaments 3042 serve as a tension support and resist elongation of the catheter wall under tension (e.g., when the catheter is being proximally retracted through tortuous vasculature).

At least one of the one or more axially extending filaments 3042 may proximally extend along the length of the catheter wall from within about 1.0 cm from the distal end of the catheter to less than about 10 cm from the distal end of the catheter, less than about 20 cm from the distal end of the catheter, less than about 30 cm from the distal end of the catheter, less than about 40 cm from the distal end of the catheter, or less than about 50 cm from the distal end of the catheter.

The one or more axially extending filaments 3042 may have a length greater than or equal to about 40 cm, greater than or equal to about 30 cm, greater than or equal to about 20 cm, greater than or equal to about 10 cm, or greater than or equal to about 5 cm.

At least one of the one or more axially extending filaments 3042 may extend at least about the most distal 50 cm of the length of the catheter, at least about the most distal 40 cm of the length of the catheter, at least about the most distal 30 cm or 20 cm or 10 cm of the length of the catheter.

In some implementations, the filament extends proximally from the distal end of the catheter along the length of the coil 24 and ends proximally within about 5 cm or 2 cm or less either side of the transition 3011 between the coil 3024 and the braid 3010. The filament may end at the transition 3011, without overlapping with the braid 3010.

In another embodiment, the most distal portion of the catheter 10 may comprise a durometer of less than approximately 35D (e.g., 25D) to form a highly flexible distal portion of the catheter and have a length between approximately 25 cm and approximately 35 cm. The distal portion may comprise one or more tubular segments of the same durometer (e.g., segment 3038). A series of proximally adjacent tubular segments may form a transition region between a proximal stiffer portion of the catheter 3000 and the distal highly flexible portion of the catheter. The series of tubular segments forming the transition region may have the same or substantially similar lengths, such as approximately 1 cm.

The relatively short length of each of the series of tubular segments may provide a steep drop in durometer over the transition region. For example, the transition region may have a proximal tubular segment 3036 (proximally adjacent the distal portion) having a durometer of approximately 35D. An adjacent proximal segment 3034 may have a durometer of approximately 55D. An adjacent proximal segment 3032 may have a durometer of approximately 63D. An adjacent proximal segment 3030 may have a durometer of approximately 72D.

More proximal segments may comprise a durometer or durometers greater than approximately 72D and may extend to the proximal end of the catheter or extension catheter segment. For instance, an extension catheter segment may comprise a proximal portion greater than approximately 72D between about 1 cm and about 3 cm. In some embodiments, the proximal portion may be about 2 cm long. In some embodiments, the most distal segments (e.g., 3038-3030) may comprise PEBAX™ and more proximal segments may comprise a generally stiffer material, such as Vestamid®.

The inner diameter of the catheter 10 may be between approximately 0.06 and 0.08 inches, between approximately 0.065 and 0.075 inches, or between 0.068 and 0.073 inches. In some embodiments, the inner diameter is approximately 0.071 inches.

In some embodiments, the distal most portion may taper to a decreased inner diameter as described elsewhere herein. The taper may occur approximately between the distal highly flexible portion and the transition region (e.g., over the most proximal portion of the distal highly flexible portion). The taper may be relatively gradual (e.g., occurring over approximately 10 or more cm) or may be relatively steep (e.g., occurring over less than approximately 5 cm). The inner diameter may taper to an inner diameter between about 0.03 and 0.06 inches. For example, the inner diameter may be about 0.035 inches, about 0.045 inches, or about 0.055 inches at the distal end of the catheter 3000. In some embodiments, the inner diameter may remain constant, at least over the catheter extension segment.

In some embodiments, the coil 3024 may extend proximally from a distal end of the catheter 10 along the highly flexible distal portion ending at the distal end of the transition region. In other embodiments, the coil 3024 may extend from a distal end of the catheter to the proximal end of the transition region, to a point along the transition region, or proximally beyond the transition region. In other embodiments, the coil 3024 may extend the entire length of the catheter 10 or catheter extension segment as described elsewhere herein. The braid 3010, when present, may extend from the proximal end of the coil 3024 to the proximal end of the catheter 10.

The one or more axially extending filaments 3042 may be placed near or radially outside the tie layer 3012 or the inner liner 3014. The one or more axially extending filaments 3042 may be placed near or radially inside the braid 3010 and/or the coil 3024. The one or more axially extending filaments 3042 may be carried between the inner liner 3014 and the helical coil 3024.

When more than one axially extending filaments 3042 are placed in the catheter wall, the axially extending filaments 3042 may be placed in a radially symmetrical manner. For example, the angle between the two axially extending filaments 3042 with respect to the radial center of the catheter may be about 180 degree. Alternatively, depending on desired clinical performances (e.g., flexibility, trackability), the axially extending filaments 3042 may be placed in a radially asymmetrical manner. The angle between any two axially extending filaments 3042 with respect to the radial center of the catheter may be less than about 180 degree, less than or equal to about 165 degree, less than or equal to about 150 degree, less than or equal to about 135 degree, less than or equal to about 120 degree, less than or equal to about 105 degree, less than or equal to about 90 degree, less than or equal to about 75 degree, less than or equal to about 60 degree, less than or equal to about 45 degree, less than or equal to about 30 degree, less than or equal to about 15 degree, less than or equal to about 10 degree, or less than or equal to about 5 degree.

The one or more axially extending filaments 3042 may be made of materials such as Kevlar, Polyester, Meta-Para-Aramide, or any combinations thereof. At least one of the one or more axially extending filaments 3042 may comprise a single fiber or a multi-fiber bundle, and the fiber or bundle may have a round or rectangular cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more axially extending filaments 3042, as measured in the radial direction, may be no more than about 2%, 5%, 8%, 15%, or 20% of that of the catheter 10.

The cross-sectional dimension of the one or more axially extending filaments 3042, as measured in the radial direction, may be no more than about 0.001 inches, about 0.002 inches, about 0.004 inches, about 0.006 inches, about 0.008 inches, or about 0.015 inches.

The one or more axially extending filaments 3042 may increase the tensile strength of the distal zone of the catheter before failure under tension to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

Any of the catheters disclosed herein, whether or not an axial filament is included, may be provided with an angled distal tip. Referring to FIG. 3D, distal catheter tip 3110 comprises a tubular body 3112 which includes an advance segment 3114, a marker band 3116 and a proximal segment 3118. An inner tubular liner 3120 may extend throughout the length of the distal catheter tip 3110, and may comprise dip coated PTFE .

A reinforcing element 3122 such as a braid or spring coil is embedded in an outer jacket 3124 which may extend the entire length of the distal catheter tip 3110.

The advance segment 3114 terminates distally in an angled face 3126, to provide a leading side wall portion 3128 having a length measured between the distal end 3130 of the marker band 3116 and a distal tip 3132. A trailing side wall portion 3134 of the advance segment 3114, has an axial length in the illustrated embodiment of approximately equal to the axial length of the leading side wall portion 3128 as measured at approximately 180 degrees around the catheter from the leading side wall portion 3128. The leading side wall portion 3128 may have an axial length within the range of from about 0.1 mm to about 5 mm and generally within the range of from about 1 to 3 mm. The trailing side wall portion 3134 may be at least about 0.1 or 0.5 or 1 mm or 2 mm or more shorter than the axial length of the leading side wall portion 3128, depending upon the desired performance.

The angled face 3126 inclines at an angle A within the range of from about 45 degrees to about 80 degrees from the longitudinal axis of the catheter. For certain implementations, the angle is within the range of from about 55 degrees to about 65 degrees or within the range of from about 55 degrees to about 65 degrees from the longitudinal axis of the catheter. In one implementation the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105%, and no more than about 130%, in some implementations within the range of from about 110% and about 125% and in one example is about 115%.

In the illustrated embodiment, the axial length of the advance segment is substantially constant around the circumference of the catheter, so that the angled face 3126 is approximately parallel to the distal surface 3136 of the marker band 3116. The marker band 3116 has a proximal surface approximately transverse to the longitudinal axis of the catheter, producing a marker band 3116 having a right trapezoid configuration in side elevational view. A short sidewall 3138 is rotationally aligned with the trailing side wall portion 3134, and has an axial length within the range of from about 0.2 mm to about 4 mm, and typically from about 0.5 mm to about 2 mm. An opposing long sidewall 3140 is rotationally aligned with the leading side wall portion 3128. Long sidewall 3140 of the marker band 3116 is generally at least about 10% or 20% longer than short sidewall 3138 and may be at least about 50% or 70% or 90% or more longer than short sidewall 3138, depending upon desired performance. Generally the long sidewall 3140 will have a length of at least about 0.5 mm or 1 mm and less than about 5 mm or 4 mm.

The marker band may have at least one and optionally two or three or more axially extending slits throughout its length to enable radial expansion. The slit may be located on the short sidewall 3138 or the long sidewall 3140 or in between, depending upon desired bending characteristics. The marker band may comprise any of a variety of radiopaque materials, such as a platinum/iridium alloy, with a wall thickness preferably no more than about 0.003 inches and in one implementation is about 0.001 inches.

The marker band zone of the assembled catheter will have a relatively high bending stiffness and high crush strength, such as at least about 50% or at least about 100% less than proximal segment 18 but generally no more than about 200% less than proximal segment 3118. The high crush strength may provide radial support to the adjacent advance segment 3114 and particularly to the leading side wall portion 3128, to facilitate the functioning of distal tip 3132 as an atraumatic bumper during transluminal advance and to resist collapse under vacuum. The proximal segment 3118 preferably has a lower bending stiffness than the marker band zone, and the advance segment 3114 preferably has even a lower bending stiffness and crush strength than the proximal segment 3118.

The advance segment 3114 may comprise a distal extension of the outer jacket 3124 and optionally the inner liner 3120, without other internal supporting structures distally of the marker band 3116. Outer jacket may comprise extruded Tecothane. The advance segment 3114 may have a bending stiffness and radial crush stiffness that is no more than about 50%, and in some implementations no more than about 25% or 15% or 5% or less than the corresponding value for the proximal segment 3118.

A support fiber 3142 as has been discussed elsewhere herein extends through at least a distal portion of the length of the proximal segment 3118. As illustrated, the support fiber 3142 may terminate distally at a proximal surface of the marker band 3116 and extend axially radially outwardly of the tubular liner 3120 and radially inwardly from the support coil 3122. Alternatively, the marker band may be provided with at least one or two axially extending slots, and the fiber can extend into the slot, thus axially overlapping with the marker band. Fiber 3142 may extend substantially parallel to the longitudinal axis, or may be inclined into a mild spiral having no more than 10 or 7 or 3 or 1 or less complete revolutions around the catheter along the length of the spiral. The fiber may comprise a high tensile strength material such as a multifilament yarn spun from liquid crystal polymer such as a Vectran multifilament LCP fiber.

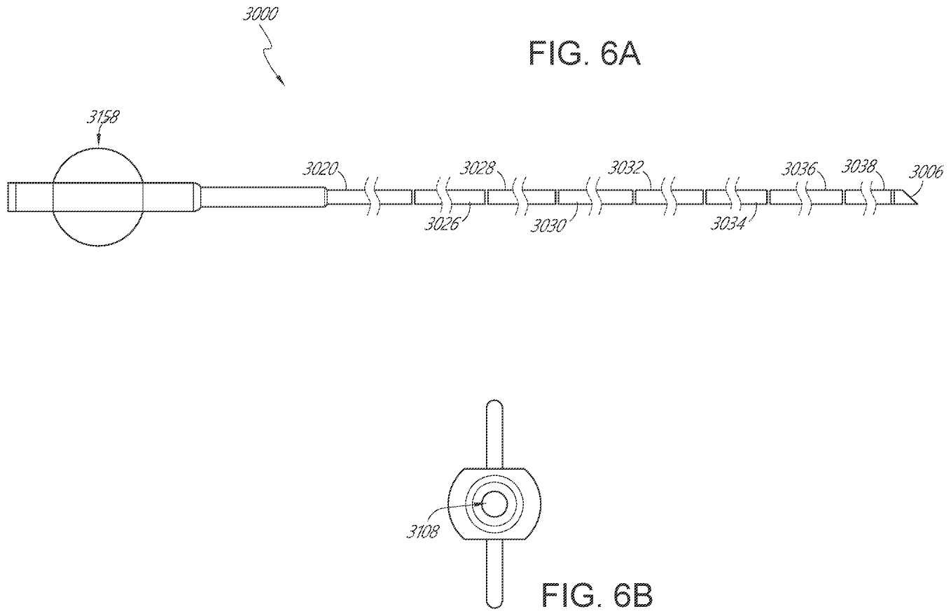

Referring to FIGS. 6A-6B, there is illustrated one example of an outer jacket segment stacking pattern for a progressive flexibility catheter of the type discussed in connection with FIG. 2. A distal segment 3038 may have a length within the range of about 1-3 cm, and a durometer of less than about 35D or 30D. An adjacent proximal segment 3036 may have a length within the range of about 4-6 cm, and a durometer of less than about 35D or 30D. An adjacent proximal segment 3034 may have a length within the range of about 4-6 cm, and a durometer of about 35D or less. An adjacent proximal segment 3032 may have a length within the range of about 1-3 cm, and a durometer within the range of from about 35D to about 45D (e.g., 40D). An adjacent proximal segment 3030 may have a length within the range of about 1-3 cm, and a durometer within the range of from about 50D to about 60D (e.g., about 55D). An adjacent proximal segment 3028 may have a length within the range of about 1-3 cm, and a durometer within the range of from about 35D to about 50D to about 60D (e.g., about 55D). An adjacent proximal segment 3026 may have a length within the range of about 1-3 cm, and a durometer of at least about 60D and typically less than about 75D. More proximal segments may have a durometer of at least about 65D or 70D. The distal most two or three segments may comprise a material such as Tecothane, and more proximal segments may comprise PEBAX or other catheter jacket materials known in the art. At least three or five or seven or nine or more discrete segments may be utilized, having a change in durometer between highest and lowest along the length of the catheter shaft of at least about 10D, preferably at least about 20D and in some implementations at least about 30D or 40D or more.

What is claimed is:

1. A catheter having jointless transitions in flexibility, comprising:
an elongate, flexible tubular body, having a proximal end, a distal end and a tubular side wall defining at least one lumen extending axially therethrough;
a helical wire coil in the side wall, the coil having a first zone in which the wire has a first radial dimension separated by a transition from a second zone in which the wire has a second radial dimension;
wherein the first zone, the transition and the second zone are formed from a single, continuous wire.

2. The catheter of claim 1, wherein the first radial dimension is a diameter.

3. The catheter of claim 1, wherein the transition has an axial length of no more than about 2 cm.

4. The catheter of claim 1, wherein the transition has an axial length of no more than about 1 cm.

5. The catheter of claim 1, wherein the lumen has the same inside diameter in the proximal zone and the distal zone.

6. The catheter of claim 1, wherein the transition is located within about 5 cm from the distal end of the catheter.

7. The catheter of claim 6, comprising at least a first jointless transition and a second jointless transition in the helical wire coil.

8. The catheter of claim 2, wherein the wire in the first zone has a diameter that is at least about 0.0002 inches greater than the diameter of the wire in the second zone.

9. The catheter of claim 8, wherein the wire in the first zone has a diameter that is at least about 0.0004 inches greater than the diameter of the wire in the second zone.

10. The catheter of claim 1, formed by the process of winding a wire coil to form a tubular body, and thereafter exposing a portion of the tubular body to a pickling agent.

11. The catheter of claim 10, wherein the pickling agent comprises an acid solution.

12. The catheter of claim 1, formed by the process of exposing a portion of a wire a pickling agent, and thereafter winding the wire into a tubular body.
